(19) United States
(12) Patent Application Publication
(10) Pub. No.: US 2016/0354023 A1
(43) Pub. Date: Dec. 8, 2016

(54) DELIRIUM DETECTION SYSTEM AND METHOD

(71) Applicant: KONINKLIJKE PHILIPS N.V., EINDHOVEN (NL)

(72) Inventors: Thomas FALCK, Aachen (DE); Esther Marjan VAN DER HEIDE, Hertogenbosch (NL); Adrienne HEINRICH, Den Bosch (NL); Yingrong XIE, Eindhoven (NL)

(21) Appl. No.: 14/730,262

(22) Filed: Jun. 4, 2015

(51) Int. Cl. A61B 5/16; A61B 5/0205; A61B 5/00; A61B 7/00; A61B 5/0402; A61B 5/0484; A61B 3/113

(52) U.S. Cl. CPC A61B 5/162; A61B 5/04842; A61B 5/04845; A61B 5/02055; A61B 3/113; A61B 7/00; A61B 5/0402; A61B 5/0077; A61B 5/0531

(57) ABSTRACT

A delirium detection system for automatic, unobtrusive and reliable detection of delirium of a person comprises a processor and a computer-readable storage medium, wherein the computer-readable storage medium contains instructions for execution by the processor. The instructions cause the processor to perform the steps of controlling a stimulus unit to output a stimulus for stimulating the person, receiving and evaluating one or more reaction signals captured by a reaction unit and reflecting the person's reaction to a stimulus, and determining a delirium score from said evaluation of one or more reaction signals.

### CROSS REFERENCE TO RELATED APPLICATIONS

This application claims the benefit of U.S. provisional application Ser. No. 62/013,030 filed Jun. 17, 2014, which is incorporated herein by reference.

### FIELD OF THE INVENTION

The present invention relates to a delirium detection system and method.

### BACKGROUND OF THE INVENTION

Delirium is a neuropsychiatric syndrome with a multifactorial etiology that occurs in a majority of ICU (Intensive Care Unit) patients. Delirium is associated with increased mortality, prolonged hospital stay, and long term effects such as decreased independent living, increased rate of institutionalization and increased risk to develop long-term cognitive impairment. Longer hospital stay and complications associated with delirium in the ICU lead to significantly higher costs of care. The prevalence rates of delirium in an ICU range from 11% to 87%. Delirium affects up to 80% ICU patients, doubles ICU mortality, triples 6-month mortality, increases length of stay by 5-10 days, involves a 9 times higher risk of permanent cognitive impairment, and attributes to $145 billion estimated annual costs in the US alone. Accurate and early detection and treatment of delirium is the key to improving patient outcome and curbing delirium-related health care costs.

Currently, for the diagnosis of delirium in ICU patients a number of validated screening questionnaires (such as the Confusion Assessment Method for ICUs (CAM-ICU)) are used. With these methods patients are checked at most three times a day, i.e. at most once per shift of caregivers. With the fluctuating character of delirium the delirious episodes are easily missed. Although it is a serious problem, delirium in patients (or persons in general) is commonly under-detected. Besides, under-detection of delirium is still the case even if screening instruments are used. Accurate and early detection methods may lead to more effective application of appropriate clinical interventions, leading to better outcome and reduced induced mortality.

WO 2013/050912 A1 discloses a monitoring system and a corresponding monitoring method for monitoring a patient and detecting delirium of the patient in an unobtrusive manner without the need of on-body sensors. The proposed monitoring system comprises a monitoring unit for obtaining image data of the patient over time, an image analysis unit for detecting motion events of the patient from the obtained image data, an evaluation unit for classifying the detected motion events into delirium-typical motion events and non-delirium-typical motion events, and a delirium determination unit for determining a delirium score from the duration, intensity, type, location and/or occurrence of delirium-typical motion events, said delirium score indicating the likelihood and/or strength of delirium of the patient.

There is a need for a continuous, objective and sensitive way for detecting delirium of persons, e.g. of ICU patients, without necessarily requiring the detection of delirium-typical motion events.

### SUMMARY OF THE INVENTION

It is an object of the present invention to provide delirium detection systems and methods for detecting delirium of persons, such as ICU-patients, in a more continuous, objective and sensitive way.

In a first aspect of the present invention a delirium detection system is presented, the system comprising a processor and a computer-readable storage medium, wherein the computer-readable storage medium contains instructions for execution by the processor, wherein the instructions cause the processor to perform the steps of:
controlling a stimulus unit to output a stimulus for stimulating the person,
receiving and evaluating one or more reaction signals captured by a reaction unit and reflecting the person's reaction to a stimulus, and
determining a delirium score from said evaluation of one or more reaction signals.

In a further aspect of the present invention a delirium detection method is presented, the method comprising the steps of:
controlling a stimulus unit to output a stimulus for stimulating the person,
receiving and evaluating one or more reaction signals captured by a reaction unit and reflecting the person's reaction to a stimulus, and
determining a delirium score from said evaluation of one or more reaction signals.

In still a further aspect of the present invention another delirium detection system is presented, the system comprising:
a controller configured to control a stimulus unit to output a stimulus for stimulating the person,
an evaluator configured to receive and evaluate one or more reaction signals captured by a reaction unit and reflecting the person's reaction to a stimulus, and
a scoring unit configured to determine a delirium score from said evaluation of one or more reaction signals.

In yet further aspects of the present invention, there are provided a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed method, computer program and medium have similar and/or identical preferred embodiments as the claimed system and as defined in the dependent claims.

The presented systems and method have the advantage that they can be used to detect delirium of a person automatically. In particular, no manual execution of the screening (e.g. according to CAM-ICU) by caregivers is required. Further, the present invention enables close meshed delirium monitoring of all patients, provides a more standardized and objective procedure, represents an assessment of delirium that is less awkward for nurses, reduces the workload of caregivers, and addresses the problem that caregivers are often reluctant to execute a screening (e.g. because they “feel silly and stupid” to ask these questions over and over again). The detected delirium score indicates the likelihood and/or strength of delirium of the patient and can be used to issue an alarm or notification, e.g. to a caregiver or a central monitoring station, if e.g. the delirium score exceeds a predetermined threshold or an individual threshold set or learned for a particular person over time.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a system according to the present invention,

FIG. 2 shows a schematic diagram of a first embodiment of a method according to the present invention,

FIG. 3 shows a schematic diagram of a second embodiment of a system according to the present invention, and

FIG. 4 shows a schematic diagram of a third embodiment of a system according to the present invention.

### DETAILED DESCRIPTION OF THE INVENTION

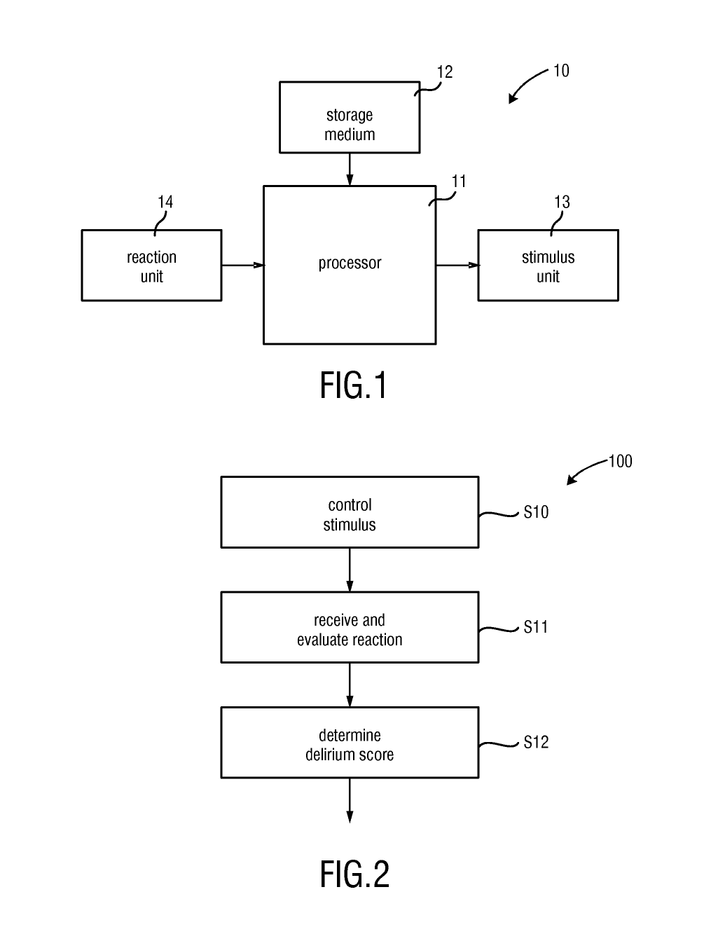

FIG. 1 shows a schematic diagram of a first embodiment of a delirium detection system 10 according to the present invention and FIG. 2 shows a schematic diagram of a first embodiment of a corresponding a delirium detection method 100 according to the present invention. The system 10 comprises a processor 11 and a computer-readable storage medium 12, wherein the computer-readable storage medium 12 contains instructions for execution by the processor 11. The instructions cause the processor 11 to perform the steps of the method 100 shown in FIG. 2. In a first step S10 a stimulus unit 13 is controlled to output a stimulus for stimulating a person. In a second step S11 one or more reaction signals captured by a reaction unit 14 and reflecting the person's reaction to a stimulus are received and evaluated. In a third step S12 a delirium score is determined from said evaluation of one or more reaction signals.

It shall be noted in this context that according to the first embodiment the system 10 comprises the computer-readable storage medium 12 and the processor 11, which outputs signals to an external stimulus unit 13 and receives signals from an external reaction unit 14. In other embodiments the system includes the stimulus unit 13 and/or the reaction unit 14. Further, whenever reference is made to a patient herein, it shall be understood that the same steps or means can be used with respect to any person and not just a patient.

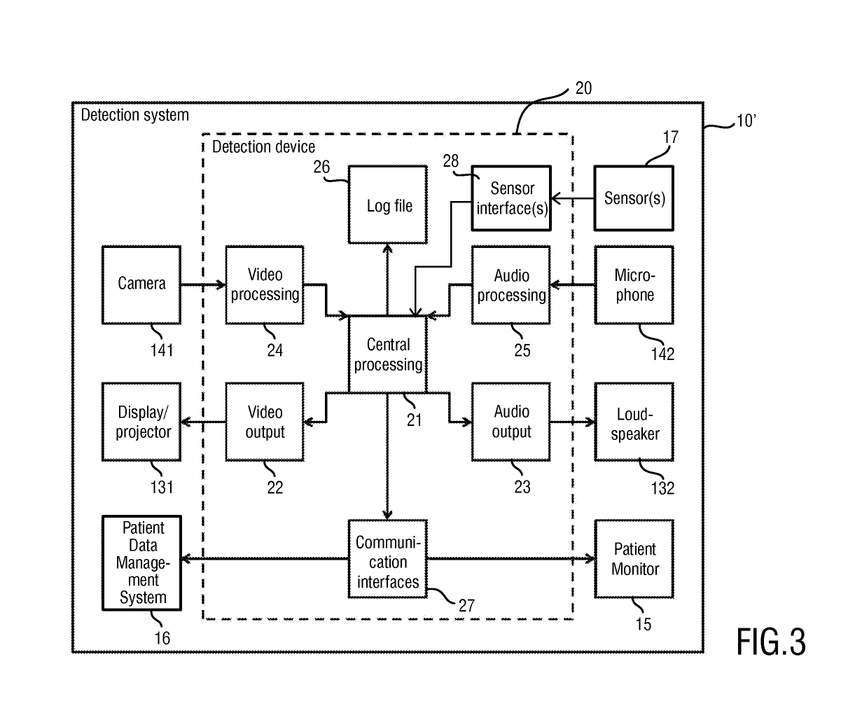

FIG. 3 shows a more detailed embodiment of a delirium detection system 10′ according to the present invention comprising a delirium detection device 20, which may be implemented as one or more processors (e.g. as a single processor 11 shown in FIG. 1) or one or more computers carrying about a corresponding computer program. The delirium detection device 20 interfaces with external components of the system 10′.

The central processing unit 21 executes a test for detecting delirium, e.g. a test similar or identical to the CAM-ICU test. For each test it creates a stimulus for the patient and evaluates the patient's reaction. It uses a video output unit 22 and external display(s) 131 or projector(s) (not shown), representing a visual stimulus unit, for rendering graphical content such as text, graphics, pictures or videos. Similarly it uses an audio output unit 23 and external loudspeaker(s) 132 or a headphone (not shown), representing an audio stimulus unit, for rendering audio content such as human speech, sounds or music. The reaction of the patient to these audio-visual stimuli are received by means of a video processing unit 24 and an audio processing unit 25 coupled to an external camera 141, representing a video reaction unit, and an external microphone 142, representing an audio reaction unit, respectively.

The central processing device 20 calculates based on the performance of the patient a delirium score which is preferably stored in a log file 26. In addition, via wired or wireless communication interfaces 27, it preferably notifies a patient monitor 15 and a patient data management system 16 about the result which in turn will notify a caregiver in case the patient turned into delirium, i.e. the delirium score indicates that the patient shows delirium or that there is a high likelihood that the patient shows delirium.

The proposed delirium detection system can e.g. be used to test the features “inattention” and “disorganized thinking” of the CAM-ICU test in an automatic way.

One example of an inattention test procedure is as follows. The central processing unit 21 instructs the audio output unit 23 to play the following recorded instruction: “I am going to read you a series of 10 letters. Whenever you hear the letter ‘A’, indicate by raising your hand. Please raise your hand if you are ready to start.” The central processing unit 21 waits until the video processing unit 24 has detected that the patient has raised his hand. Then it instructs the audio output unit 23 to read the following recorded letters at a rate of one letter every 3 seconds: “S” “A” “V” “E” “H” “A” “A” “R” “T”. The video processing unit 24 informs the central processing unit 21 whenever it has detected that the patient has raised his hand. The central processing unit 21 counts the errors, i.e. when a patient did not raise his hand on the letter “A” and when the patient raised his hand on any letter other than “A”. From these counts inattention of the patient and a delirium score can be determined, e.g. by use of a threshold for the number of counts of wrongly raising the hand or not raising the hand.

Another example of an inattention test procedure is as follows. The central processing unit 21 instructs the audio output unit 23 to play the following recorded instruction: “I am going to show you pictures of some common objects. Watch carefully and try to remember each picture because I will ask what pictures you have seen. Let me know when you saw a picture before by raising your hand. Please raise your hand if you are ready to start.” The central processing unit 21 waits until the video processing unit 24 has detected that the patient has raised his hand. Then it instructs the video output unit 22 to show 5 pictures randomly selected from a picture database for three seconds each and in parallel the audio output unit 23 to name each item, e.g. dog, boot, knife, brush, pants, etc. Then the central processing unit 21 instructs the audio output unit 23 to play the following recorded instructions: “Now I am going to show you some more pictures. Some of these you have already seen and some are new. Let me know when you saw a picture before by raising your hand. Please raise your hand if you are ready to start.” The central processing unit 21 waits until the video processing unit 24 has detected that the patient has raised his hand. Then it instructs the visual output unit 22 to show 10 pictures (5 new +5 repeat) in random order from a picture database for three seconds each and in parallel the audio output unit 23 to name each item, e.g. dog, boot, knife, brush, pants, etc. The video processing unit 24 informs the central processing unit 21 whenever it has detected that the patient has raised his hand. The central processing unit 21 counts the errors, i.e. when a patient did not raise his hand on a repeated picture and when the patient raised his hand on any new picture.

One example of a disorganized thinking test procedure is as follows. The central processing unit 21 instructs the audio output unit 23 to play the following recorded instruction: “I am going to ask you four yes/no questions. Let me know the answers by nodding your head yes or no. Please raise your hand if you are ready to start.” The central processing unit 21 waits until the video processing unit 24 has detected that the patient has raised his hand. Then it instructs the audio output unit 23 to read the following recorded questions: “Will a stone float on water?”, “Are there fish in the sea?”, “Does one pound weigh more than two pounds?”, and “Can you use a hammer to pound a nail?”. The video processing unit 24 informs the central processing unit 21 whenever it has detected that the patient has nodded his head. The central processing unit 21 counts the errors, i.e. when a patient nodded yes on a no question and when the patient nodded no on a yes question.

Another example of a disorganized thinking test procedure is as follows. The central processing unit 21 instructs the video output unit 22 to show a picture of a hand holding up 2 fingers and the audio output unit 23 to play the following recorded instruction: “Hold up this many fingers”. The central processing unit 21 waits until the video processing unit 24 has detected that the patient has raised his hand and counts the number of fingers held up. The video processing unit 24 informs the central processing unit 21 how many fingers the patient held up. The central processing unit 21 counts the errors, i.e. when a patient did not hold up two fingers. Then the central processing unit 21 instructs the audio output unit 23 to play the following recorded instruction: “Now do the same thing with the other hand”. The central processing unit 21 waits until the video processing unit 24 has detected that the patient has raised his hand and counts the number of fingers held up. The video processing unit 24 informs the central processing unit 21 how many fingers the patient held up. The central processing unit 21 counts the errors, i.e. when a patient did not hold up two fingers.

Optionally, caregivers will be notified if the patient is detected as being suspicious for delirium, e.g. have more than two errors in inattention and more than one error is disorganized thinking.

Various modifications are possible according to further embodiments. In particular:
Visual output can be replaced by verbal output, and vice versa.
Visual input can be replaced by verbal input, and vice versa.
Other gestures can be used for yes/no answers, e.g. eye blinking, nodding, lip moving, etc. Generally, any kinds of gestures or mimics, eye blinking, eye movement, head movement, lip movement and/or movement of one or more other body parts may be monitored and used as input signal for the evaluation and detection if the person has a delirium.
Instead of letters in an inattention procedure e.g. balls in different colors can be rendered by the video output unit and the patient has to raise his hand (or otherwise signal) whenever he sees e.g. a blue ball on the screen.
To test inattention the patient can be asked to count the number of balls rendered on the screen.
To test inattention moving objects can be rendered on the screen and the camera monitors the patient's head movement to detect if the patient is losing track.
To test inattention moving/travelling sound can be rendered and the system monitors the patient's eye movement to detect if the patient is losing track.
Inattention can be also tested in combination with EEG sensor or other sensors 17, whose sensor signals are provided to the central processing unit via a sensor interface 28. For instance, an oddball object, e.g. a fish suddenly showing up in a sky full of birds, can be rendered. If the patient is attentive, then there will be a peak in the EEG signal when the oddball object appears. If no change in the EEG signal happens when the oddball object appears, then that indicates inattention of the patient.
Other sensors may be one or more of an ECG sensor, a heart rate sensor, a breathing rate sensor, a blood pressure sensor, a temperature sensor, a skin conductivity sensor and/or a body movement sensor. Generally, any kinds of vital signs may be monitored and used as input signal for the evaluation and detection if the person has a delirium. Vital signs may also be obtained and monitored by a camera recording a sequence of images from which PPG signals are derived and which are used to extract vital signs such as heart rate or breathing rate using the remote photo-plethysmography technique.
An LED screen or other light equipment, as e.g. provided on a ceiling of a patient room, can be used as display for stimulation.
Generally, a monitor, a TV set, a light apparatus and/or a beamer may be used as visual stimulus unit and a loudspeaker is preferably used as an audio stimulus unit.

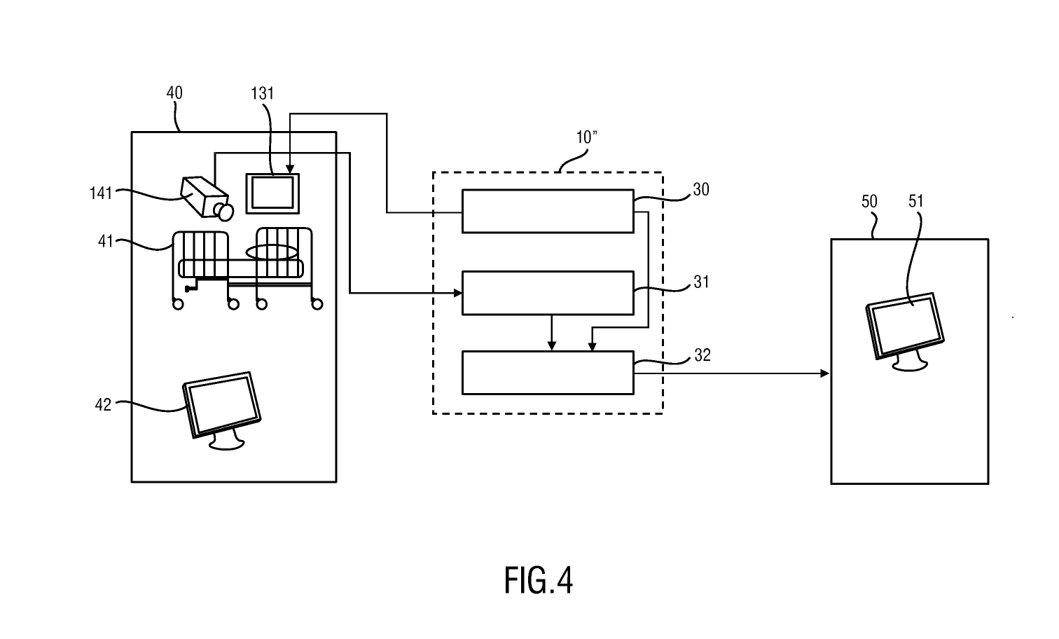

FIG. 4 shows a schematic diagram of a third embodiment of a delirium detection system 10″ according to the present invention. In this embodiment the system 10″ comprises a controller 30 configured to control a stimulus unit 131 (in this embodiment a monitor) to output a stimulus for stimulating the person, in this embodiment a patient lying in a bed 41, e.g. in a patient room 40. An evaluator 31 is provided to receive and evaluate one or more reaction signals captured by a reaction unit 141, in this embodiment a camera mounted above the bed for monitoring the patient. Said reaction signals reflect the person's reaction to a stimulus. A scoring unit 32 is provided to determine a delirium score from said evaluation of one or more reaction signals.

The elements 30 to 32 may be provided in the patient room 40 as well, but are preferably at a separate location, e.g. in a nursing station or central monitoring room of a station in a hospital. The generated data, in particular the delirium score, is displayed on a bedside patient monitor 42 and/or a nurse monitor 51, preferably included in an overall overview of all patients that can be assessed at the nursing station 50.

The present invention can be applied to person monitoring in general, in particular for automatic detection of delirious patients in hospitals, in particular ICUs, PACUs, and geriatric wards, but also in nursing homes, and senior living facilities.

In the claims, the word “comprising” does not exclude other elements or steps, and the indefinite article “a” or an does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible device or apparatus that can contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, can be coupled to the system either directly or through intervening I/O controllers. These devices may include, for example, without limitation, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

The description of the different illustrative embodiments has been presented for purposes of illustration and description and is not intended to be exhaustive or limited to the embodiments in the form disclosed. Many modifications and variations will be apparent to those of ordinary skill in the art. Further, different illustrative embodiments may provide different advantages as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated. Other variations to the disclosed embodiments can be understood and effected by those skilled in the art in practicing the claimed invention, from a study of the drawings, the disclosure, and the appended claims.

1. A delirium detection system for detecting delirium of a person, the system comprising a processor and a computer-readable storage medium, wherein the computer-readable storage medium contains instructions for execution by the processor, wherein the instructions cause the processor to perform the steps of:
controlling a stimulus unit to output a stimulus for stimulating the person,
receiving and evaluating one or more reaction signals captured by a reaction unit and reflecting the person's reaction to a stimulus, and
determining a delirium score from said evaluation of one or more reaction signals.

2. The system as claimed in claim 1, wherein the instructions further cause the processor to control a visual and/or audio stimulus unit to generate and output a visual and/or audio stimulus.

3. The system as claimed in claim 1, wherein the instructions further cause the processor to receive and evaluate visual, audio and/or vital sign reaction signals captured by corresponding reaction units.

4. The system as claimed in claim 1, wherein the instructions further cause the processor to output a notification if the delirium score indicates or suggests delirium of the person.

5. The system as claimed in claim 1, wherein the instructions further cause the processor to control a visual stimulus unit to show pictures or to show objects at changing positions.

6. The system as claimed in claim 1, wherein the instructions further cause the processor to control an audio stimulus unit to issue sounds, read text and/or ask questions.

7. The system as claimed in claim 1, wherein the instructions further cause the processor to receive and evaluate visual reaction signals captured by a camera showing the person's reaction to a stimulus.

8. The system as claimed in claim 7, wherein the instructions further cause the processor to receive and evaluate gestures, mimics, eye blinking, eye movement, head movement, lip movement and/or movement of one or more other body parts.

9. The system as claimed in claim 7, wherein the instructions further cause the processor to control a stimulus unit to issue a moving object and/or a moving sound, to receive eye movement signals and/or head movement signal and to evaluate if the person loses track of the moving object and/or moving sound.

10. The system as claimed in claim 7, wherein the instructions further cause the processor to control a visual stimulus unit to show an oddball object within a series of non-oddball objects and to receive and evaluate a reaction signal reflecting the person's response to the showing of the oddball object.

11. The system as claimed in claim 1, wherein the instructions further cause the processor to receive and evaluate audible reaction signals captured by a microphone recording the person's audible reaction or answer to a stimulus.

12. The system as claimed in claim 1, wherein the instructions further cause the processor to receive and evaluate vital sign reaction signals captured by one or more vital sign measurement units recording one or more vital signs of the person in reaction to a stimulus.

13. The system as claimed in claim 12, wherein the instructions further cause the processor to receive and evaluate the person's EEG signal, ECG signal, heart rate signal, breathing rate signal, blood pressure signal, temperature signal, skin conductivity signal and/or body movement signal.

14. The system as claimed in claim 1, wherein the instructions further cause the processor to output a stimulus at regular or irregular intervals.

15. The system as claimed in claim 1, further comprising a visual stimulus unit for outputting a visual stimulus including a monitor, a TV set, a light apparatus and/or a beamer and/or an audio stimulus unit for outputting an audible stimulus including a loudspeaker.

16. The system as claimed in claim 1, further comprising by a reaction unit including a camera, a microphone and/or one or more sensors.

17. The system as claimed in claim 1, wherein said reaction unit includes one or more of an EEG sensor, an ECG sensor, a heart rate sensor, a breathing rate sensor, a blood pressure sensor, a temperature sensor, a skin conductivity sensor and/or a body movement sensor.

18. A delirium detection method for detecting delirium of a person, the method comprising the steps of:
controlling a stimulus unit to output a stimulus for stimulating the person,
receiving and evaluating one or more reaction signals captured by a reaction unit and reflecting the person's reaction to a stimulus, and
determining a delirium score from said evaluation of one or more reaction signals.

19. A non-transitory computer-readable recording medium that stores therein a computer program product, which, when executed by a processor, causes the method as claimed in claim 18 to be performed.

20. A delirium detection system for detecting delirium of a person, the system comprising:
a controller configured to control a stimulus unit to output a stimulus for stimulating the person,
an evaluator configured to receive and evaluate one or more reaction signals captured by a reaction unit and reflecting the person's reaction to a stimulus, and
a scoring unit configured to determine a delirium score from said evaluation of one or more reaction signals.
